(19) United States
(12) Patent Application Publication
(10) Pub. No.: US 2004/0186353 A1
(43) Pub. Date: Sep. 23, 2004

(54) Vascular retractor and methods of using same

(76) Inventor: Thomas J Palermo, San Jose, CA (US)

Correspondence Address: Douglas E Denninger, U S Surgical a division of, Tyco Healthcare Group, 150 Glover Avenue, Norwalk, CT, 06856, US

(21) Appl. No.: 10/485,766

(22) PCT Filed: Aug. 9, 2002
(86) PCT No.: PCT/US02/25503

Related U.S. Application Data
(60) Provisional application No. 60/311,374, filed on Aug. 10, 2001.

(51) Int. Cl. A61B 1/32

(52) U.S. Cl. 600/201

(57) ABSTRACT

Vascular retractor assemblies and methods for retracting and maintaining a subcutaneous working space at a surgical site are disclosed, the assemblies including an external frame, a retractor pad assembly including a retractor pad configured and dimensioned to contact a surface of the surgical site, an adjustment mechanism and at least one port provided in the lower surface of the retractor pad. The at least one port is preferably in fluid communication with the surface of the surgical site. In one embodiment, the at least one port is in fluid communication with a source of medical adhesive for adhering or creating a bond between the surface of the surgical site to the retractor pad. In another embodiment, the at least one port is in fluid communication with a source of vacuum for selectively adhering or creating a bond between the surface of the surgical site to the retractor pad.

### CROSS-REFERENCE TO RELATED APPLICATIONS

The present application claims priority of U.S. Provisional Patent Application Serial No. 60/311,374 filed Aug. 10, 2001, the entire contents of which are incorporated herein by reference.

### BACKGROUND

1. Technical Field

The present disclosure relates to surgical retractors and their methods of use, and more particularly to vascular retractors that are self-supporting and provide a longitudinal working window for endoscopic vascular harvesting procedures and their methods of use.

2. Background

Numerous surgical procedures have been developed to replace or bypass arteries that have become blocked by disease. Aortocoronary bypass surgery is perhaps the most important of these surgical procedures. The coronary arteries supply blood to the heart. As a result of aging and disease, coronary arteries may become blocked by plaque deposits, stenosis, or cholesterol. In some instances, these blockages can be treated with artherectomy, angioplasty or stent placement, and coronary bypass surgery is not required. Coronary bypass surgery is required when these other methods of treatment cannot be used or have failed to clear the blocked artery. In coronary bypass surgery, a blood vessel is harvested from elsewhere in the body and grafted into place between the aorta and the coronary artery beyond the point of blockage.

The coronary bypass surgery requires a length of blood vessel or artery for the graft. It is preferred to use a blood vessel taken from the patient undergoing the bypass surgery. The patient is a ready source of suitable blood vessels that will not be rejected by the body after transplantation and grafting onto the aorta and coronary artery. The saphenous vein in the leg is the best substitute for small arteries such as the coronary arteries, and it is the preferred blood vessel for use in coronary bypass surgery. This is because the saphenous vein is typically 3 to 5 mm in diameter, i.e., about the same size as the coronary arteries. Also, the venous system of the legs is sufficiently redundant so that, after removal of the saphenous vein, other blood vessels that remain in the leg are adequate to provide return blood flow. The cephalic vein in the arm is an alternative that is sometimes used.

Previously an operation to harvest the saphenous vein required the surgeon to make an incision into the leg in order to gain access to the saphenous vein and then proceed to cut the vein from the leg. In order to expose the vein, the surgeon made a series of incisions generally from the groin to the knee or the ankle leaving one or more skin bridges along the line of the incisions. It was recommended that handling of the saphenous vein be kept to a minimum while the saphenous vein was removed from the surrounding connective tissue. After exposing the saphenous vein, the surgeon grasped the saphenous vein with his fingers while stripping off the surrounding tissues with dissecting scissors or other scraping instruments. The surgeon would use his fingers and/or blunt dissection tools to pull and lift (or mobilize) the vein from the surrounding tissue. The saphenous vein was mobilized or pulled as far as possible through each incision. To reach under the skin bridges, the surgeon lifted the skin with retractors and dug the saphenous vein free. While stripping the vein, the surgeon would encounter the various tributary blood vessels that feed into the saphenous vein. These tributaries needed to be ligated and divided. To divide and ligate the tributaries that lay under the skin bridges, the surgeon needed to cut one end of the saphenous vein and pull it under the skin bridge to gently pull the saphenous vein out from under the skin bridge until the tributary was sufficiently exposed so that it could be ligated and divided. In certain instances surgeons made one continuous incision from the groin to the knee and/or ankle. The surgeon proceeded to pull and lift the saphenous vein from the surrounding tissue and when the saphenous vein was completely mobilized, the surgeon cut the proximal and distal ends of the saphenous vein and removed it from the leg. After removal, the saphenous vein was prepared for implantation into the graft site, and the long incisions made in the leg are stitched closed.

More recently, vein harvesting has been accomplished using endoscopic procedures. In the endoscopic procedure, one or more small incisions are made at selected target sites for providing access to the vein being harvested. For example, to harvest the saphenous vein, an incision may be made at the groin, at the knee, and/or at the ankle. A tunneling instrument, such as a blunt or soft-tipped dissector may be utilized to dissect a subcutaneous space along the anterior surface of the vein being harvested. Such tunneling instruments generally include a substantially transparent elongate member having a rounded distal end and a passage therein for receiving an endoscope which provides visualization through the end and/or side walls of the dissector.

The tunneling instrument is inserted into the incision and advanced or pushed along between tissue layers to identify the saphenous vein. The tip of the dissector is generally kept in contact with the vein and the dissector is advanced along the tissues, thereby creating a small tunnel along the anterior surface of the vein. An inflatable balloon may then be introduced into the tunnel (or alternatively provided in a collapsed condition on the tunneling instrument prior to insertion into the incision), and inflated to enlarge and further propagate the tunnel. The balloon may be used to dissect fat and skin overlying the vein and to enlarge the tunnel to a desired size. Other means, including further blunt dissection may also be used to enlarge the tunnel.

Once the desired length of vein is exposed and an appropriate tunnel developed, the balloon and/or dissector is/are removed, and a retractor, typically a wide flat shaft with a handle on its proximal end, is inserted into the incision and directed along the dissected path over the section of vein to be harvested. The handle of the retractor may then be lifted away from the surface of the leg, creating a space under the shaft adjacent the vein.

Surgical instruments, such as a vein harvesting hook, may then be inserted into the space to strip away tissues surrounding the vein, ligate tributary veins, and mobilize the vein. Typically, the retractor has substantially transparent walls and an endoscope is provided in a passage in the retractor, thereby allowing visualization during the harvesting procedure.

The retraction devices, such as those used in the vein harvesting procedure just described, typically require external support to hold the retractor away from the surface of the vein and maintain the anatomic space. The surgeon may have to hold the handle of the retractor, preventing both hands from being free for the procedure or the surgeon may require an assistant for performing the procedure. Alternatively, an external mechanical support may be provided to hold the retractor.

Some retractors include a distal hood capable of maintaining a space thereunder. These hoods, however, only create a limited self-supported space, requiring that the retractor be moved when it is desired to work in a new location. Such retractors also generally require external support to provide a space along the retractor shaft between the incision and the hooded space.

As a response to some of these limitation, new devices and techniques have been developed including retractors having a substantially rigid elongate member, having proximal and distal ends, and having a tunnel or an arcuate, arch shaped or “C” shaped cross-section configured to hold the dissected space open after being inserted into the dissected space. The cross-section of the elongate member defines a “C” shaped passage extending distally from the proximal end, and provides a longitudinal working window along the passage between the longitudinal edges of the arch, that is, below the longitudinal terminal edges. Such a system is shown in U.S. Pat. No. 6,228,024, the disclosure of which is incorporated herein by reference in its entirety. Other systems are shown in U.S. Pat. Nos. 6,068,039 and 6,196,968, the disclosures of which are incorporated herein by reference in their entireties.

Although such a “C-shaped” retractor offers the advantage of providing a retracted working space adjacent the target vessel, it is desirable to have a less intrusive means of retracting a working space that eliminates unnecessary structures that can obscure the surgical field. Moreover, it would be desirable to have a retractor including means for improving visualization without the need for inserting a device into the working space.

Accordingly, the need exists for a retractor capable of holding open an anatomic space for endoscopic vascular procedures that improves visualization without obscuring the surgical field. In addition, the need exists for a retractor for holding open an anatomic space for endoscopic procedures that provides improved visualization within the space while eliminating the need for inserting a device into the working space.

### SUMMARY

The present disclosure is directed to surgical retractors for harvesting blood vessels from the body. The present disclosure is also directed to methods of using the surgical retractors of the present disclosure to perform the vessel harvesting procedure.

In accordance with one aspect of the present disclosure, a vascular retractor assembly for retracting and maintaining a subcutaneous working space at a surgical site includes an external frame, a retractor pad assembly including a retractor pad configured and dimensioned to contact a surface of the surgical site, an adjustment mechanism and at least one port provided in the lower surface of the retractor pad. The retractor pad assembly is preferably adjustably couplable to the external frame. The adjustment mechanism is operatively coupled between the external frame and the retractor pad assembly with the adjustment mechanism being configured and adapted to adjust the position of the retractor pad assembly relative to the external frame. The at least one port is preferably in fluid communication with the surface of the surgical site.

In one embodiment, the at least one port is in fluid communication with a source of medical adhesive for adhering the surface of the surgical site to the retractor pad, while in another embodiment, the at least one port is in fluid communication with a source of vacuum for selectively adhering the surface of the surgical site to the retractor pad.

The external frame preferably includes a pair of inverted substantially U-shaped support members and a base member interconnecting the corresponding distal ends of each U-shaped support member to one another. Each U-shaped support member includes a pair of uprights interconnected at a proximal end thereof by a bridge portion with each bridge portion having an elongated slot formed therein.

The retractor pad assembly preferably includes a pair of brackets mounted to an upper surface of the retractor pad. Each bracket is configured and adapted to be slidably received through a respective elongated slot formed in the bridge portions of the U-shaped support members. Preferably, each bracket is substantially rectangular having a pair of spaced apart sidewalls interconnected by an upper and a lower wall. The pair of side walls of each bracket are configured and dimensioned to be slidably received through a respective elongated slot of the U-shaped support members and the lower wall of each bracket is secured to the upper surface of the rectangular pad.

The adjustment mechanism includes a threaded body extending through the upper wall of each bracket such that a distal surface of each threaded body contacts an upper surface of the bridge portion of a respective U-shaped support member. Accordingly, a rotation of the threaded body of the adjustment mechanism results in the retractor pad assembly moving distally or proximally with respect to the external frame.

The retractor pad can be shaped to approximate the contours of the surface of the surgical site. In one embodiment, the retractor pad has a minor axis and a major axis and the retractor pad is curved along a minor axis thereof.

Preferably, the source of medical adhesive includes a first reservoir for a first component of a medical epoxy and a second reservoir for a second component of the medical epoxy. Accordingly, the medical adhesive is formed upon a mixing together of the first and second components of the medical epoxy with one another.

In an alternative embodiment, the distal ends of each respective U-shaped support member are configured and adapted to rest against the surface of the surgical site. Preferably, the distal ends of each respective U-shaped support member diverge from one another.

The present disclosure also provides a method of retracting and maintaining a subcutaneous working space at a surgical site. The preferred method includes creating a subcutaneous working space proximate the surgical site, providing a retractor assembly including an external frame and a retractor pad operatively coupled to the external frame, the retractor pad including at least one port formed on a bottom surface thereof, creating a retraction force along a bottom surface of the retractor pad via the at least one port, contacting the bottom surface of the retractor pad to the surface of the skin overlying the subcutaneous working space in order to engage the bottom surface of the retractor pad to the surface of the skin overlying the subcutaneous working space and adjusting the position of the retractor assembly to retract the skin overlying the subcutaneous working space and to maintain the subcutaneous working space.

According to one method the retraction force is created by providing a medical adhesive to the bottom surface of the retractor pad, while according to another method, the retraction force is created by providing a vacuum to the bottom surface of the retractor pad.

The preferred method further provides for the external frame of the retractor assembly to include a pair of inverted substantially U-shaped support members. Each U-shaped support member including a pair of uprights interconnected at a proximal end thereof by a bridge portion. Each bridge portion having an elongated slot formed therein and a base member interconnecting a corresponding distal end of each U-shaped support member to one another.

The preferred method further provides for the retractor assembly to include a pair of brackets mounted to an upper surface of the retractor pad. Each bracket is configured and adapted to be slidably received through a respective elongated slot formed in the bridge portions of the U-shaped support members and an adjustment mechanism for adjusting the position of the retractor pad with respect to the external frame.

Other objects and features of the present disclosure will become apparent from consideration to the following description taken in conjunction with the accompanying drawings.

### BRIEF DESCRIPTION OF THE DRAWINGS

The accompanying drawings, which are incorporated in and constitute a part of this specification, illustrate embodiments of the disclosure and, together with the general description given above, and the detailed description of the embodiments given below, serve to explain the principles of the present disclosure.

FIG. 1 is a side elevational view of a vascular retractor assembly, in accordance with an embodiment of the present disclosure;

FIG. 2 is a side elevational view of a retractor pad assembly of the vascular retractor assembly of FIG. 1, in accordance with an embodiment of the present disclosure;

FIG. 3 is an end view of the retractor pad assembly of FIG. 2;

FIG. 4 is a top plan view of a retractor pad assembly of FIG. 2;

FIG. 5 is a bottom plan view of the retractor pad assembly of FIG. 2;

FIG. 6 is a perspective view of a leg illustrating the use of the vascular retractor assembly of FIG. 1 to facilitate a saphenous vein harvesting procedure;

FIG. 7 is a cut-away end view of the vascular retractor assembly of FIG. 1 disposed about a patient's leg and retracting a working space proximate the saphenous vein;

FIG. 8 is a top plan view of the vascular retractor assembly of FIG. 1 disposed about the patient's leg and proximate the saphenous vein;

FIG. 9 is cut-away end view of a vascular retractor assembly, in accordance with an alternative configuration of the present disclosure, disposed about a patient's leg and retracting a working space proximate the saphenous vein; and

FIG. 10 is a cut-away end view of a vascular retractor assembly, in accordance with another alternative configuration of the present disclosure, disposed about a patient's leg and retracting a working space proximate the saphenous vein.

### DETAILED DESCRIPTION OF PREFERRED EMBODIMENTS

Preferred embodiments of the presently disclosed blood vessel harvesting device will now be described in detail with reference to the drawing figures wherein like reference numerals identify similar or identical elements. In the drawings and in the description which follows, the term “proximal”, as is traditional will refer to the end of the surgical device or instrument of the present disclosure which is closest to the operator, while the term “distal” will refer to the end of the device which is furthest from the operator.

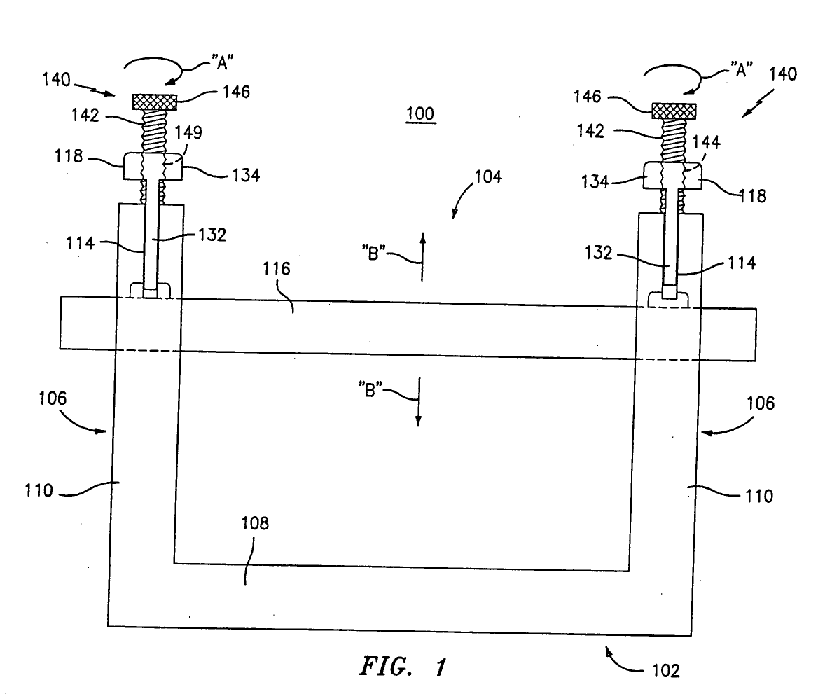

Referring now to the drawings, wherein like reference numerals identify similar or identical parts throughout the views, and particularly to FIGS. 1-5, a vascular retractor assembly, in accordance with the present disclosure, is shown generally as reference numeral 100. Vascular retractor assembly 100 includes an external frame 102 and a retractor pad assembly 104 operatively couplable to frame 102.

As seen in FIGS. 1 and 6-8, frame 102 includes a pair of inverted substantially “U-shaped” support members 106 interconnected by a pair of base members 108 extending between the corresponding distal ends of each “U-shaped” support member 106. “U-shaped” support members 106 are sized to accommodate an anatomical structure, such as, for example, a patient's thigh, while leaving an open space sufficient to access a surgical site in the anatomical structure. Each “U-shaped” support member 106 includes a pair of spaced apart uprights 110 interconnected by a bridge portion 112. Each bridge portion 112 includes a longitudinal elongate slot 114 formed therein. As will be described in detail below, elongate slots 114 permit retractor pad assembly 104 to be slid along slots 114 so that a retractor pad may be precisely positioned relative to the anatomical structure.

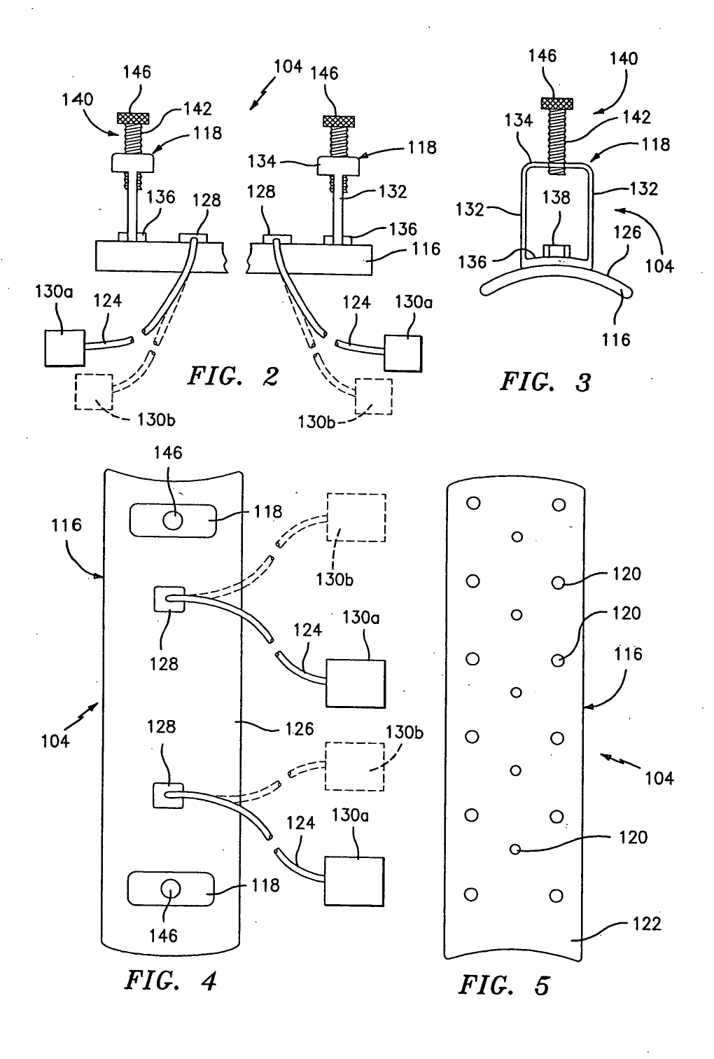

As seen in FIGS. 1-8 and, in particular, in FIG. 2-5, retractor pad assembly 104 includes a retractor pad 116 and, preferably, at least a pair of brackets 118 operatively coupled to an upper surface thereof. Preferably, retractor pad 116 is a substantially flattened elongate member having a slight curvature which approximates the anatomical topography in the vicinity of the body structure which is to be harvested. By way of example, for saphenous vein harvesting applications, it is contemplated that retractor pad 116 can be curved along its minor axis to approximate the curvature of the patient's leg in the region of the portion of the saphenous vein to be harvested. Alternative shapes and geometries for retractor pad 116 are envisioned which approximate the geometries of various harvesting locations. Such alternative shapes are largely determined by the geometry of the body in the vicinity of the body structure to be harvested.

Retractor pad 116 is preferably substantially stiff and can be made from plastic, stainless steel or other materials well-known in the medical field. Alternatively, retractor pad 116 can be substantially flexible and include a longitudinal support pocket (not shown) running the length thereof. In this embodiment, a set of substantially stiff interchangeable support inserts (not shown) can be provided, which set of interchangeable support inserts approximate the various contours of the anatomical structure against which they are to contact.

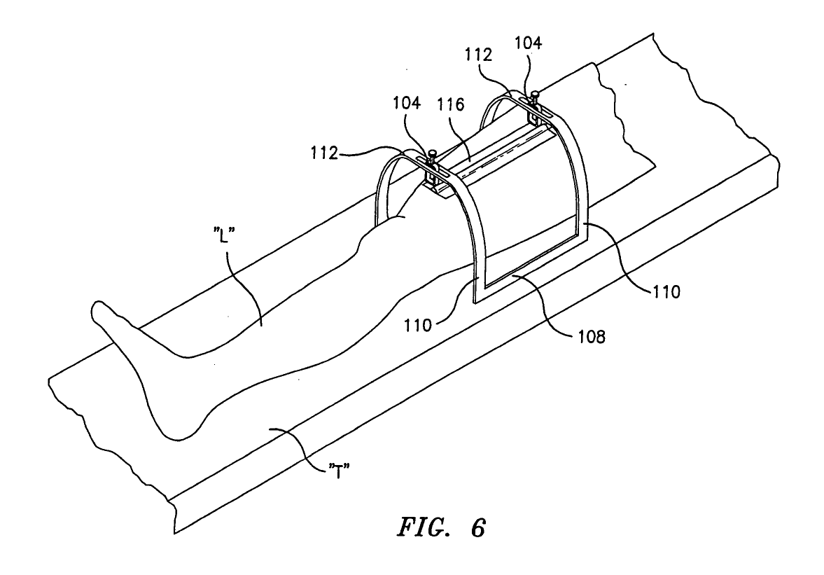

As seen in FIG. 5, retractor pad 116 includes a plurality of ports 120 formed in a bottom surface 122 thereof for engaging a surface of the patient's skin. Preferably, as seen in FIGS. 2 and 4, the plurality of ports 120 are in fluid communication, via at least one tubing 124 operatively coupled to a top surface 126 of retractor pad 116 through a hub 128, with a source of vacuum as is schematically represented by block 130a. Alternatively, in another embodiment, the plurality of ports 120 are in fluid communication, via tubing 124 and hub 128, with a source of medical adhesive as is also schematically represented by block 130b.

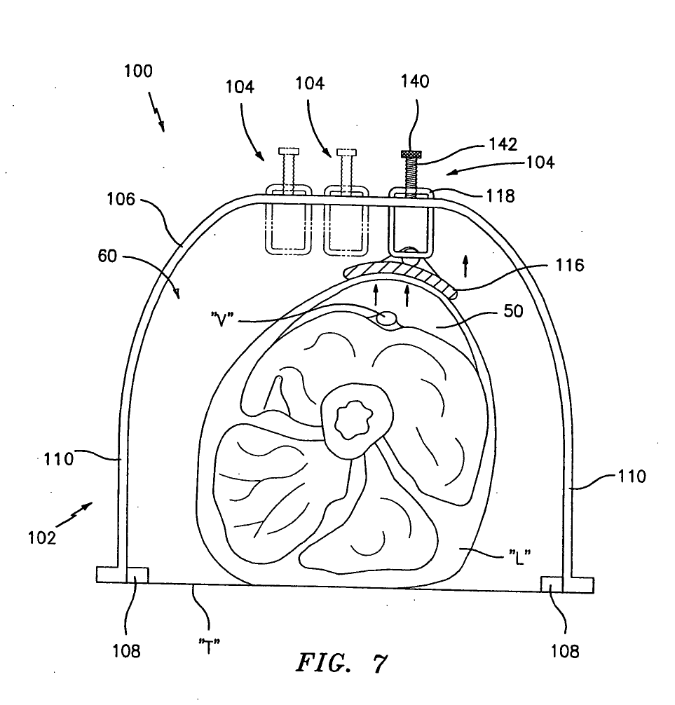

As seen in particular in FIG. 3, each bracket 118 is substantially rectangular in shape having a pair of spaced apart substantially parallel side walls 132 interconnected by an upper and a lower wall member 134, 136, respectively. While brackets 118 are shown as substantially rectangular, it is envisioned that brackets 118 can be “L-shaped”, “U-shaped”, circular or any other shape depending on the application for which retractor assembly 100 is intended. Side walls 132 are configured and dimensioned to be slidably received through slot 114 of frame 102. As seen in FIG. 7, brackets 118 can be positioned anywhere along the length of elongate slot 114. While a pair or brackets 118, provided near a proximal and a distal end of retractor pad 116 is preferred, it is envisioned that retractor assembly 100 can include a single bracket 118 positioned near the site to be incised or retracted.

Brackets 118 are fastened to retractor pad 116 via a nut and bolt arrangement 138. Preferably, retractor pad 116 includes a pair of threaded bolts extending from upper surface 126 of retractor pad 116. The pair of threaded bolts are preferably spaced from one another a distance which is substantially equal to the distance between slots 114 of frame 102. Lower wall member 136 of each bracket 118 is in turn provided with a through hole (not shown) for receiving a respective bolt therethrough. A nut can then be attached to the end of the bolt extending through lower wall member 136 to thereby couple retractor pad 116 to each bracket 118. It is contemplated that each bracket 118 may be integrally incorporated with retractor pad 116 to increase the ease of manufacture and to improve the resterilization of retractor assembly 100.

Each bracket 118 is further provided with an adjustment mechanism 140 for adjusting the relative distance of retractor pad 116 with respect to bridge portion 112 of “U-shaped” support member 106 of frame 102. Adjustment mechanism 140 includes a threaded screw portion 142, threadably receivable within a threaded hole 144 formed in upper wall 134 of each bracket 118, and a head portion 146 provided at a proximal end thereof. Preferably, screw portion 142 is dimensioned to have a diameter larger than the width of elongate slot 114 of frame 102. Accordingly, in use, a distal surface of screw portion 142 will contact an upper surface of bridge portion 112 of “U-shaped” support member 106 of frame 102.

In this manner, when retractor pad assembly 104 is assembled with frame 102, retractor pad assembly 104 depends from (i.e., hangs down from or is suspended from) frame 102. Accordingly, as seen in FIG. 1, rotation of screw portion 142, as indicated by arrow “A”, will cause retractor pad assembly 104 to linearly move (i.e., rise or fall depending on the direction of rotation), as indicated by arrows “B”, with respect to bridge portion 112 of frame 102. In particular, a clockwise rotation of screw portion 142 will cause bracket 118 to move proximally with respect to frame 102 while a counterclockwise rotation of screw portion 142 will cause bracket 118 to move distally with respect to frame 102. It is contemplated that other adjustment mechanisms can include a pulley/cable arrangement, a multi-bar linkage or a hydraulic system.

Alternatively, retractor pad assembly 104 can be adjustable relative to frame 102 via a cooperative tabs and recess arrangement therefore. One example of such a system is shown in U.S. Pat. No. 6,196,968, the entire contents of which have previously been incorporated by reference. In an alternative embodiment, it is envisioned that screw portion 142 threadably passes through bridge portion 112 of frame 102 in order to prevent movement of retractor pad assembly 104 in a proximal direction. Other adjustment means are also suitable for use with the present disclosure without departing from the spirit and scope of the present disclosure.

A preferred method of use of a vascular retractor assembly, in accordance with the present disclosure, will now be described with reference to FIGS. 1-8 and, in particular to FIGS. 6-8, for use in a saphenous vein harvesting procedure. It should be understood, however, that the methods disclosed herein are applicable to harvesting procedures for other body structures as well.

In accordance with a preferred method, a subcutaneous space is initially incised or dissected using conventional methods. Alternatively, U.S. Pat. No. 5,601,581 to Fogarty et al., the entire disclosure of which is incorporated herein by reference, discloses an apparatus and method suitable for dissecting a subcutaneous space. According to the method disclosed in Fogarty et al., a section of a tissue structure, for example a nerve of a vein, especially the saphenous vein, may be selected to be harvested. An incision is created at a location adjacent to one end of the selected structure, such as at the groin or the knee. A tunneling instrument, such as a blunt or a soft-tipped dissector, possibly including an inflatable balloon thereon, may be inserted into the incision and advanced along between tissue layers to identify the selected tissue structure. The tunneling instrument may be advanced along the anterior surface of the tissue structure and/or a balloon on the tunneling instrument may be inflated to create a dissected space of a desired size. Once the desired dissected space is developed, the balloon may be deflated, and the tunneling instrument may be removed from the dissected space through the incision.

Preferably, after the subcutaneous space has been created, vascular retractor assembly 100, including frame 102 and retractor pad assembly 104 are positioned proximate a leg “L” of the patient such that retractor pad 116 of retractor pad assembly 104 overlies the area of the patient's skin which corresponds to the subcutaneous space created and uprights 110 lay substantially along the sides of leg “L”. In one preferred method, surgical adhesive is then provided to the skin/pad interface via ports 120 in order to create a solid bond between the skin of the patient and inner surface 122 of retractor pad 116. Suitable medical adhesives are well known in the art and may include a fast drying epoxy that bonds when two constituent components of the epoxy are mixed together. For example, retractor assembly 100 can include a first reservoir for a first component of a medical epoxy and a second reservoir for a second component of a medical epoxy. As such, the medical adhesive (i.e., medical epoxy) is formed upon a mixing together of the first and second components of the medical adhesive. In other words, adhesive sources 130 may be configured to each provide one constituent component of the medical epoxy that is to be mixed and used to bond to the surface of the skin. Also, desirable adhesion characteristics can be provided by currently marketed adhesives for disposable ECG- or RF-dispersive pads.

It is envisioned that the surgeon may directly apply medical adhesive, preferably a water soluble medical adhesive, to the surface of retractor pad 116, thereby eliminating the need for ports 120, tube 124 and adhesive source 130. It is further envisioned that ports 120 may be used solely for the application of an adhesive deactivator, solvent or water to allow more rapid removal of retractor pad assembly 104 from the surface of the skin to which it is adhered.

In an alternative method, it is envisioned that if a retractor assembly 100 having a source of vacuum 130 is used, a vacuum can be provided to the skin/pad interface via ports 120 in order to create a suction-type bond between the skin of the patient and inner surface 122 of retractor pad 116. The vacuum is preferably user-controlled so that the operator can increase or decrease the vacuum pressure. Although it is desirable to have a secure skin/retractor-pad interface, it is recommended that excessive pressure is avoided to prevent inadvertent hematoma formation. It is contemplated that tubes 124 may also include a stop valve (not shown) to cut or modify pressure when desired. Vacuum offers the added benefits of allowing near instantaneous affixing and removal of retractor pad assembly 104 to and from the surface of the skin adjacent to the working space.

Once a suitable skin/retractor-pad bond has been created, either via adhesives or a vacuum, retractor pad 116 is retracted away from the surface of the leg by, for example, rotating screw portion 142 of adjustment mechanism 140. In this manner, the skin overlying the vein to be harvested, i.e., the saphenous vein, is pulled away from the vein to increase the working space in order to enable the vein to be dissected and ligated along the extent of its length.

In order to increase the force that may be applied by the retractor, leg “L” of the patient may be strapped, taped or otherwise held down in contact with the operating table to prevent leg “L” from being raised off of the table.

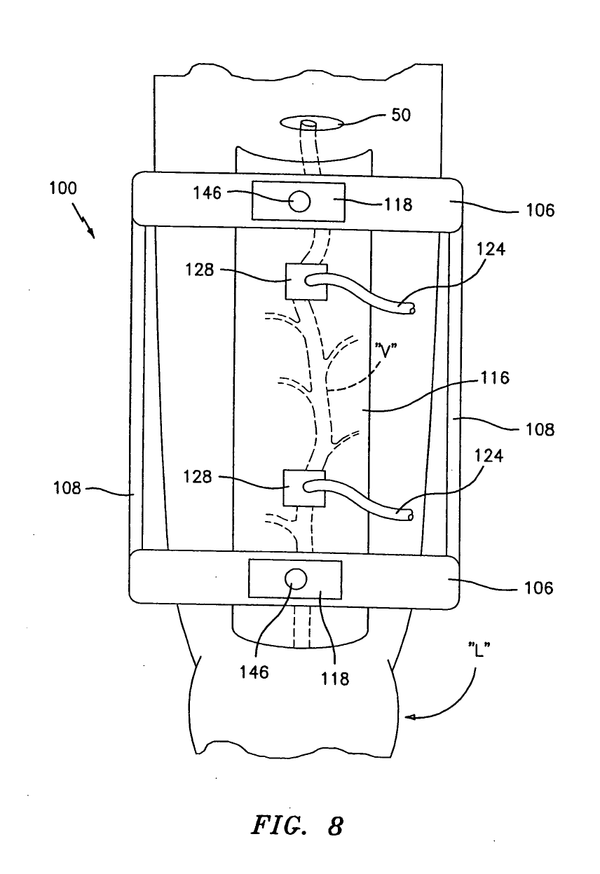

After a suitable working space has been created and maintained by retractor assembly 100, the surgical harvesting procedure may proceed through an incision 50 (see FIGS. 7 and 8) made in leg “L” and the opening 60 defined by “U-shaped” support member 106 of frame 102.

After completion of the harvesting procedure, the harvested saphenous vein “V” may be removed via incision 50 and the tension on retractor pad 116, overlying the skin, released by counter-rotating screw portion 142 of adjustment mechanism 140 to thereby lower retractor pad assembly 104 relative to the surface of the skin. After retractor pad 116 has been lowered, in the case of a retractor assembly 100 using an adhesive interface between retractor pad 116 and the surface of the skin, the adhesive may be deactivated using a deactivating agent that can be applied directly to the skin/retractor-pad interface by the surgeon or may be supplied to the interface via tubes 124 through ports 120. Such deactivating agents are known and function to reduce or eliminate the binding capabilities of the medical adhesive and allow removal of retractor assembly 100.

If an additional length of vein is desired to be harvested, retractor assembly 100 may be adjusted by being moved along the leg a desired distance and the above procedure repeated at a second incision site. Thus, a retractor assembly 100, in accordance with the principles of the present disclosure, will allow a relatively long section of vein, nerve or other tissue structure to be harvested through a series of relatively small incisions spaced apart from one another. Once the tissue structure has been removed from the incision, retractor assembly 100 is removed and the skin incisions are closed.

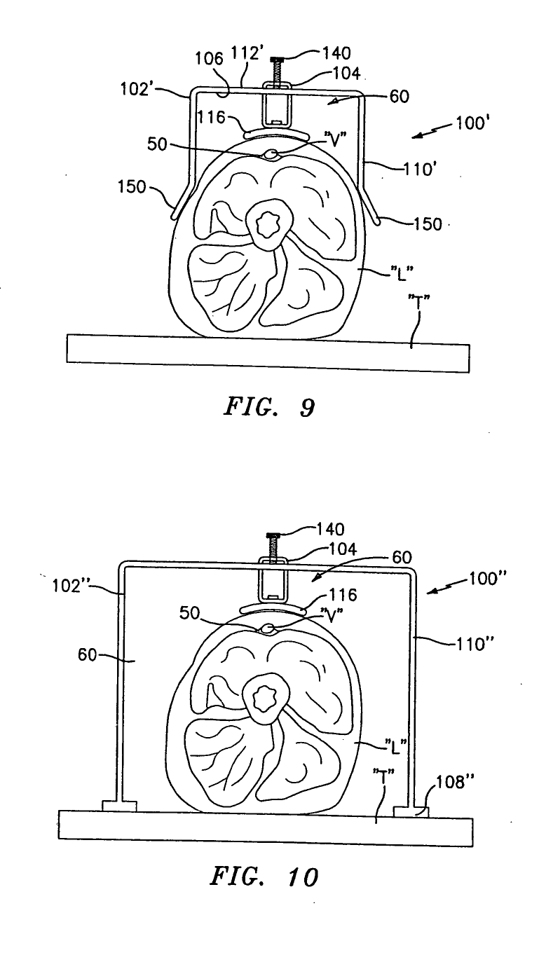

Turning now to FIGS. 9 and 10, alternate configurations of an external frame are shown. In FIG. 9, external frame 102′ is configured to be braced against the body of the patient, such as, for example, the leg of the patient. In particular, “U-shaped” support member 106′ of external frame 102′ includes a bridge portion 112′ having a length which is less than a width of the patient's leg “L” and wherein spaced apart uprights 110′ have diverging distal ends 150 which are configured and adapted to rest against the surface of the patient's leg “L”. In other words, no part of external frame 102′ is in contact with operating table “T” on top of which the patient lies.

In FIG. 10, external frame 102″ is configured and adapted to be fixedly attached to or integral with operating table “T”. It is contemplated that the distal ends of uprights 110″ can be adapted to receive screws or bolts (not shown) therethrough, which screw or bolt engage a corresponding threaded hole formed in the surface of operating table “T”. It is further contemplated that external frame 102″ can be secured to the surface of operating table “T” by a plurality of straps (not shown) wrapped around base members 108″ and are tied to operating table “T”. Other securing means are also suitable for use with the present apparatus without departing from the spirit or scope of the disclosure.

While the disclosure is susceptible to various modifications, and alternative forms, specific examples thereof have been shown in the drawings and are herein described in detail. It should be understood, however, that the disclosure is not to be limited to the particular forms or methods disclosed, but to the contrary, the disclosure is to cover all modifications, equivalents and alternatives falling within the spirit and scope of the appended claims.

What is claimed is:

1. A vascular retractor assembly for retracting and maintaining a subcutaneous working space at a surgical site comprising:
an external frame;
a retractor pad assembly including a retractor pad configured and dimensioned to contact a surface of the surgical site, the retractor pad assembly being adjustably couplable to the external frame;
an adjustment mechanism operatively coupled between the external frame and the retractor pad assembly, the adjustment mechanism being configured and adapted to adjust the position of the retractor pad assembly relative to the external frame;
at least one port provided in a lower surface of the retractor pad, oriented toward the surface of the surgical site, the at least one port being in fluid communication with the surface of the surgical site; and
a pressure source operatively connected to the at least one port for applying a pressure to the at least one port.

2. The vascular retractor assembly according to claim 1, wherein the pressure applied to the at least one port is a positive pressure.

3. The vascular retractor assembly according to claim 2, wherein the at least one port is in fluid communication with a source of medical adhesive for adhering the surface of the surgical site to the retractor pad.

4. The vascular retractor assembly according to claim 1, wherein the pressure applied to the at least one port is a negative pressure.

5. The vascular retractor assembly according to claim 4, wherein the at least one port is in fluid communication with a source of vacuum for selectively adhering the surface of the surgical site to the retractor pad.

6. The vascular retractor assembly according to claim 1, wherein the external frame comprises:
a pair of inverted substantially U-shaped support members, each U-shaped support member including a pair of uprights interconnected at a proximal end thereof by a bridge portion, each bridge portion having an elongated slot formed therein; and
a base member interconnecting the corresponding distal ends of each U-shaped support member to one another.

7. The vascular retractor assembly according to claim 6, wherein the retractor pad assembly comprises:
a pair of brackets mounted to an upper surface of the retractor pad, each bracket configured and adapted to be slidably received within a respective elongated slot formed in the bridge portions of the U-shaped support members.

8. The vascular retractor assembly according to claim 7, wherein each bracket is substantially rectangular having a pair of spaced apart sidewalls interconnected by an upper and a lower wall, the pair of side walls of each bracket being configured and dimensioned to be slidably received through a respective elongated slot of the U-shaped support members, the lower wall of each bracket being secured to the upper surface of the rectangular pad.

9. The vascular retractor assembly according to claim 8, wherein the adjustment mechanism comprises:
a threaded body extending through the upper wall of each bracket such that a distal surface of each threaded body contacts an upper surface of the bridge portion of a respective U-shaped support member, wherein a rotation of the threaded body of the adjustment mechanism causes the retractor pad assembly to move distally or proximally with respect to the external frame.

10. The vascular retractor assembly according to claim 9, wherein the retractor pad is shaped to approximate the contours of the surface of the surgical site.

11. The vascular retractor assembly according to claim 9, wherein the retractor pad has a minor axis and a major axis and wherein the retractor pad is curved along a minor axis thereof.

12. The vascular retractor assembly according to claim 9, wherein the at least one port is in fluid communication with a source of vacuum for selectively adhering the surface of the surgical site to the retractor pad.

13. The vascular retractor assembly according to claim 9, wherein the at least one port is in fluid communication with a source of medical adhesive for adhering the surface of the surgical site to the retractor pad.

14. The vascular retractor assembly according to claim 13, wherein the source of medical adhesive includes a first reservoir for a first component of a medical epoxy and a second reservoir for a second component of the medical epoxy, wherein the medical adhesive is formed upon a mixing together of the first and second components of the medical epoxy with one another.

15. The vascular retractor assembly according to claim 9, wherein the distal ends of each respective U-shaped support member are configured and adapted to rest against the surface of the surgical site.

16. The vascular retractor assembly according to claim 15, wherein the distal ends of each respective U-shaped support member diverge from one another.

17. A method of retracting and maintaining a subcutaneous working space at a surgical site comprising the steps of:
creating a subcutaneous working space proximate the surgical site;
providing a retractor assembly including an external frame and a retractor pad operatively coupled to the external frame, the retractor pad including at least one port formed on a bottom surface thereof;
creating a retraction force along a bottom surface of the retractor pad via the at least one port;
contacting the bottom surface of the retractor pad to the surface of the skin overlying the subcutaneous working space in order to create a bond between the bottom surface of the retractor pad and the surface of the skin overlying the subcutaneous working space; and
adjusting the position of the retractor assembly to retract the skin overlying the subcutaneous working space and to maintain the subcutaneous working space.

18. The method of retracting and maintaining a subcutaneous space according to claim 17, wherein the retraction force is created by providing a medical adhesive to the bottom surface of the retractor pad.

19. The method of retracting and maintaining a subcutaneous space according to claim 17, wherein the retraction force is created by providing a vacuum to the bottom surface of the retractor pad.

20. The method of retracting and maintaining a subcutaneous space according to claim 17, wherein the external frame of the retractor assembly includes a pair of inverted substantially U-shaped support members, each U-shaped support member including a pair of uprights interconnected at a proximal end thereof by a bridge portion, each bridge portion having an elongated slot formed therein and a base member interconnecting a corresponding distal end of each U-shaped support member to one another.

21. The method of retracting and maintaining a subcutaneous space according to claim 20, wherein the retractor assembly includes a pair of brackets mounted to an upper surface of the retractor pad, each bracket is configured and adapted to be slidably received within a respective elongated slot formed in the bridge portions of the U-shaped support members and an adjustment mechanism for adjusting the position of the retractor pad with respect to the external frame.
